(19) United States
(12) Patent Application Publication
(10) Pub. No.: US 2020/0405755 A1
(43) Pub. Date: Dec. 31, 2020

(54) AST-120 for non-systemic newborn and pediatric hyperammonemia therapy

(71) Applicant: Donald N. Halgren, Manchester, MA (US)

(72) Inventor: Ronald J. Thompson, Cincinnati, OH (US)

(21) Appl. No.: 16/873,181

(22) Filed: Feb. 21, 2020

Related U.S. Application Data
(60) Provisional application No. 62/919,399, filed on Mar. 12, 2019.

(51) Int. Cl. A61K 33/44; A61K 9/16

(52) U.S. Cl. CPC A61K 33/44; A61K 9/16

(57) ABSTRACT

50% of newborns with Urea Cycle Disorders have severe hyperammonemia within the first week of life, that can, if untreated or inadequately treated, promptly cause mental retardation and death. Systemic hyperammonemia therapies are limited. The present invention teaches of the administration of AST-120 (spherical carbon absorbent) as a non-systemic therapy for the prevention and treatment of hyperammonemia of Urea Cycle Disorders in Newborns, Infants and Children.

### BACKGROUND OF THE INVENTION

The present invention relates to the treatment of urea cycle disorders in newborns and children

### Field of the Invention

The field of the present invention is the treatment of newborns, infants, and children with hyperammonemia due to urea cycle disorders, an inherited inborn error of metabolism, and is based upon Provisional Application No. 62/919,399, filed Mar. 2, 2019, which is incorporated herein by reference in its entirety.

### Prior Art

U.S. patent application Ser. No. 15/704,174 “Hyperammonemia therapy for children suffering from urea cycle disorders” Thompson, filed Sep. 14, 2017. U.S. Provisional Patent Application “Continuous non-systemic hyperammonemia therapy for newborns with urea cycle disorders” Thompson, filed Feb. 22, 2019. U.S. Pat. No. 8,110,186 “Method to maintain the efficacy of orlistat” Thompson issued Feb. 7, 2012. EP1407772 “Pharmaceutical composition comprising porous spherical carbonaceous substance and its use for treatment of renal and liver diseases” Hayushisa et al filed Sep. 9, 2002. U.S. Pat. No. 8,865,161 “Absorbent for oral administration and agent for treating or preventing renal or liver disease” Sonobe et al, filed Jun. 6, 2012. U.S. Pat. No. 9,844,568 “Porous carbon particles for use in the treatment or prevention of liver disease” Howell et al, filed Mar. 15, 2013. All of these cited references are included in the present patent application, in their entirety, by reference.
GB 2,053,176
U.S. Pat. No. 7,651,974 Sonobe et al
JP 4,311.923 Ise et al
U.S. Pat. No. 3,681,764 Endo et al
TW 1,607,765 Honda et al
U.S. Pat. No. 8,048,413 Huguet et al
U.S. Pat. No. 8,388,984 Huguet et al
References: Hyperammonemia of Urea Cycle Disorders (in addition to references cited in above Patent Applications)
US FDA AMMONUL Product Information
US FDA RAVICTI Product Information
Wasant et al Urea cycle disorders in Thai infants: a report of 5 cases. J Med Assoc Thai 2002 August; 85 Supplement 2: S 720-31
Kido et al Early liver transplantation in neonatal-onset and moderate urea cycle disorders may lead to normal development. Metab Brain Dis 2018 October; 33(5) :1517-1523
Posset et al Age at disease onset and peak ammonium level rather than interventional variables predict neurological outcome in urea cycle disorders. J Inherit Metab Dis 2016 September; 39(5): 661-672
Unsinn et al Clinical course of 63 patients with neonatal onset of urea cycle disorders in the years 2001-2013. Orphanet J Rare Dis 2016 Aug. 19; 11(1): 116
Wasibren et al Improving long term outcomes in urea cycle disorders-report from the Urea Cycle Disorders Consortium (678 UCD patients) J Inherit Metab Dis 2016 July: 39(4); 573-84
References: AST-120
Bosoi et al AST-120 (spherical carbon absorbent) lowers ammonia levels and attenuates brain edema in bile duct-ligated rats. Hepatology 2011 June, 53(6): 1995-2002

### Backround: AST-120 References By The Numbers

The US National Library of Medicine at the National Institutes of Health (PubMed) lists 239 references for AST-120.
224 of the AST-120 references report renal disease and treatment of chronic renal disease.
15 of the AST-120 references report treatment of Hepatic Encephalopathy, Crohn's Disease, Irritable Bowel Syndrome, Perianal Fistula, and Pouchitis.
All 239 AST-120 references are either Adult Human or Animal Studies reports.
None of the 239 AST-120 references included Newborns, Infants or Children.
None of the 239 AST-120 references included Hyperammonemia or Urea Cycle Disorders.

### Further Background of the Invention: AST-120 Regulatory Status and Clinical Trials

AST-120 is not US FDA approved, and never has been approved for any indication, although AST-120 has been extensively, clinically studied in the US. AST-120 is marketed as Kremezin for Chronic Kidney Disease in Japan and Korea, and has been approved and marketed for decades in Japan and Korea. ClinicalTrials.gov from the US National Library of Medicine and the National Institutes of Health, lists 17 AST-120 Clinical Trials in humans over the past 12 years. These AST-120 Clinical Trials included over 3000 patients with efficacy evaluation of Inflammatory Bowel Disease (Crohn's Disease and Ulcerative Colitis), Chronic Kidney Disease, Hepatic Encephalopathy, Pouchitis, Gastro Esophageal Reflux Disease (GERD), and Irritable Bowel Disease (IBS). None of the AST-120 Clinical Trials proved efficacy over placebo, for any disease studied. None of the AST-120 Clinical Trials included Newborns, Infants, or Children. None of the AST-120 Clinical Trials included Hyperammonemia or Urea Cycle Disorders.

### Ocera Therapeutics, Inc

AST-120 was in-licensed by Ocera Therapeutics, from Kureha Corporation, in early 2006. Ocera received the rights to Clinically develop AST-120 for treatment of Gastrointestinal and Liver diseases, with marketing rights in the US and Europe. The initial disease studied was adult mild hepatic encephalopathy affecting some 200,000 patients with liver disease.

Bosoi et al in the 2011, reported in Hepatology, “AST-120 (spherical carbon absorbent) lowers ammonia levels and attenuates brain edema in bile duct-ligated rats”. This was the required animal study for the efficacy study, NCT00867698 “AST-120 Used to Treat Mild Hepatic Encepathalopathy” (ASTUTE). Dr Bosoi reported AST-120 attenuated brain edema in hyperammonia induced rats, which allowed the ASTUTE trial. Dr Bosoi also reported that AST-120 successfully reduced ammonia acetate induced hyperammonemia, on a dose dependent basis. Untreated, 177 microM ammonia, 0.1 gm/KG/day, 121 microM ammonia, 1.0 gm/KG/day, 81 microM ammonia, and 4 gm/KG/day, 49 microM ammonia. The ASTUTE trial did not measure serum ammonia levels!!

The ASTUTE trial was a $10 million trial of 148 mildly impaired hepatic encephalopathy patients with the Primary Outcome Measurement of “Neurocognitive improvement, defined as the change in the global summary score of the RBANS at week 8 compared to baseline”. The AST-120 could not establish efficacy for AST-120 for this indication. Ocera did not file an NDA with the FDA for this indication. Ocera abandoned the study of AST-120 for hepatic encephalopathy.

### SUMMARY OF THE PRESENT INVENTION

The present invention is a method to orally administer AST-120 (spherical carbon absorbent) as a non-systemic therapy for prevention or treatment of hyperammonemia of urea cycle disorders in newborns, infants, and children.

The incidence of urea cycle disorders is generally quoted as 1:35,000 births. Urea cycle disorders are genetically inherited and present within several days after birth, or anytime usually within the first 7 years of life. The severe genetic defects present early, and the less severe genetic defects present late. The early presenting defect urea cycle diseases can have profound hyperammonemia, and lead to mental retardation or death very rapidly.

Unsinn et al in “Clinical course of 63 patients with neonatal onset of urea cycle disorders in the years 2001-2013” reports, “In about half of patients presenting with urea cycle disorders, the first symptoms appear within a few days after birth”. Dr Posset et al in “Age at disease onset and peak ammonium level rather than interventional variables predict neurological outcomes in urea cycle disorders” reports, “in 456 UCD patients about two-thirds remain asymptomatic until age 12 days (of life). Peak ammonium level of the initial hyperammonemic crisis (500 microM/L) best predicted neurological outcomes”.
Newborn=first 28 days of life
Infant=first year of life
Child=first 12 years of life

Ammonia and the ammonium ion are both neurotoxic and easily cross the blood brain barrier. Both have a molecular weight of 18, similar to water with a molecular weight of 17, and all freely diffuse from the small bowel lumen, into the bloodstream, and from the bloodstream into the brain. The normal ammonia level is about 100 microM/L. Severe mental retardation and death are associated with ammonia levels of 300-500 microM/L. Very young newborns and infants have immature livers and pancreatic lipase production, and systemic hyperammonemia therapy with oral RAVICTI and intravenous AMMONUL is less than optimum.

AMMONUL HAS A BLACK BOX WARNING

ANY EPISODE OF ACUTE SYMPTOMATIC HYPERAMMONEMIA SHOULD BE TREATED AS A LIFE-THREATENING EMERGENCY. TREATMENT OF HYPERAMMONEMIA MAY REQUIRE DIALYSIS, PREFERABLY HEMODIALYSIS, TO REMOVE A LARGE BURDEN OF AMMONIA. UNCONTROLLED HYPERAMMONEMIA CAN RAPIDLY RESULT IN BRAIN DAMAGE OR DEATH, AND PROMPT USE OF ALL THERAPIES NECESSARY TO REDUCE AMMONIA LEVELS IS ESSENTIAL.

The invention thus comprises a method of treating or preventing hyperammonemia of urea cycle disorders in newborns, infants and children, comprising the step of: administering AST-120 (spherical carbon absorbent) to the newborn, infant or child. The method may include administering the AST-120 via a nasogastric, orogastric, or percutaneous gastric feeding tube. The method may include administering a pharmaceutical grade AST-120. The method may include administering the AST-120 at least four times per day. The method may include the step of administrating AST-120 which is dosed between about 0.5 gm/KG/day and 5 gm/KG/day. The method may include administering AST-120 which is dosed at 1-2 gm/KG/day. The method may include administering AST-120 so as to prevent an ammonia level above 50 micro M/L. The method may include administering AST-120 to treat ammonia levels from about 100- to about 500 micro M/L. The method may cover wherein the hyperammonemia prevention is non-systemic. The method may include administering a dosage of AST-120 to newborns and infants.

1. A method of treating or preventing hyperammonemia of urea cycle disorders in newborns, infants and children, comprising the step of:
administering AST-120 (spherical carbon absorbent) to the newborn, infant or child.

2. The method of treating or preventing hyperammonemia of urea cycle disorders in newborns, infants and children as recited in claim 1, comprising the step of:
administering the AST-120 via a nasogastric, orogastric, or percutaneous gastric feeding tube.

3. The method of treating or preventing hyperammonemia of urea cycle disorders in newborns, infants and children as recited in claim 1, comprising the step of:
administering a pharmaceutical grade AST-120.

4. The method of treating or preventing hyperammonemia of urea cycle disorders in newborns, infants and children as recited in claim 1, comprising the step of:
administering the AST-120 at least four times per day.

5. The method of treating or preventing hyperammonemia of urea cycle disorders in newborns, infants and children as recited in claim 1, wherein the administrated AST-120 is dosed between about 0.5 gm/KG/day and 5 gm/KG/day.

6. The method of treating or preventing hyperammonemia of urea cycle disorders in newborns, infants and children as recited in claim 5, wherein the administrated Ast-120 is dosed at 1-2 gm/KG/day.

7. The method of treating or preventing hyperammonemia of urea cycle disorders in newborns, infants and children as recited in of claim 1, the method including:
administering AST-120 so as to prevent an ammonia level above 50 microM/L.

8. The method of treating or preventing hyperammonemia of urea cycle disorders in newborns, infants and children as recited in claim 1, wherein the method includes:
administering AST-120 to treat ammonia levels from about 100- to about 500 microM/L.

9. The method of treating or preventing hyperammonemia of urea cycle disorders in newborns, infants and children as recited in claim 1, where the hyperammonemia prevention is non-systemic.

10. The method of treating or preventing hyperammonemia of urea cycle disorders in newborns, infants and children as recited in claim 1, wherein the hyperammonemia treatment is non-systemic.

11. The method of treating or preventing hyperammonemia of urea cycle disorders in newborns, infants and children as recited claim 1, the method comprising:
administering a dosage of AST-120 to newborns and infants.
